(19) United States
(12) Patent Application Publication
(10) Pub. No.: US 2017/0065785 A1
(43) Pub. Date: Mar. 9, 2017

(54) HEADGEAR APPARATUS TO PREVENT OR REDUCE INJURY FROM PROLONGED CANNULA USE

(71) Applicant: Brett H. Roberts, Ogden, UT (US)

(72) Inventor: Brett H. Roberts, Ogden, UT (US)

(21) Appl. No.: 15/259,272

(22) Filed: Sep. 8, 2016

Related U.S. Application Data
(60) Provisional application No. 62/215,236, filed on Sep. 8, 2015.

(51) Int. Cl. A61M 16/06

(52) U.S. Cl. CPC A61M 16/0683; A61M 16/0666; A61M 2205/0222; A61M 2207/00

(57) ABSTRACT

Systems and methods provide cushioning to the tubing of a user-worn nasal cannula delivery system to mitigate the potentially negative effects of direct tubing-to-skin contact in the area around the user's ears. An apparatus may include multiple shaped straps individually connected to shaped hold pieces. The apparatus securely holds the cannula tubing in place while shielding the tubing from the tissues around the user's ears. In some embodiments, multiple straps may be connected to create a single headgear piece that protects both ears while resting snugly against the user's head.

### RELATED APPLICATIONS

This application claims the benefit under 35 U.S.C. §119(e) of U.S. Provisional Application No. 62/215,236, filed Sep. 8, 2015, which is hereby incorporated by reference herein in its entirety.

### TECHNICAL FIELD

This disclosure relates to an apparatus for providing a cushioning barrier to certain tubing regions of a user-worn nasal cannula delivery system to mitigate the potentially negative effects of direct tubing-to-skin contact. More specifically, the present disclosure is related to a headgear device that surrounds the tubing of a traditionally-worn nasal cannula delivery system in sensitive/affected areas around an ear of the user.

### BRIEF DESCRIPTION OF THE DRAWINGS

Non-limiting and non-exhaustive embodiments of the disclosure are described herein, including various embodiments of the disclosure with reference to the drawings described below.

FIG. 1 is a perspective view of a nasal cannula user wearing one embodiment of the apparatus of the current disclosure.

FIG. 2 is a schematic diagram of a strap of the apparatus according to one embodiment.

FIG. 3 is a schematic diagram of a hold piece according to one embodiment.

FIG. 4 is a schematic diagram of an attachment of a hold piece to a strap, the attachment occurring near respective shaped ends of the hold piece and the strap such that the respective shaped ends substantially align, according to one embodiment.

FIG. 5 is a schematic diagram of two straps and two hold pieces generally arranged in ordered positions (but before being attached to each other), with hold fasteners and adjustable strap fasteners placed in example locations on the various pieces, according to one embodiment.

FIG. 6 is a schematic diagram of the location of the hold fasteners and the adjustable strap fastener once the respective straps and hold pieces of FIG. 5 have been attached together, according to one embodiment.

FIG. 7A is a schematic diagram of the fastening of two straps in a first chosen position using an adjustable strap fastener, according to one embodiment.

FIG. 7B is a schematic diagram of the fastening of two straps in a second chosen position using an adjustable strap fastener, according to one embodiment.

FIG. 8 is a schematic diagram of a hold piece and a strap according to a second embodiment, which includes non-slip material near portions of a hold fastener on both the strap and the hold piece, according to one embodiment.

References to the figures throughout the description are for convenience only. As provided herein, embodiments of the systems and methods described herein may include one or more additional components or features not illustrated in the figures. Similarly, one or more of the illustrated components or features may be omitted and/or substituted for a different component or feature in any of the embodiments described herein. Furthermore, the described features, structures, or characteristics on any embodiment may be combined in any suitable manner in one or more alternative embodiments, including all possible permutations thereof.

### DETAILED DESCRIPTION

The present disclosure includes various systems and methods for preventing injuries related to the wear and use of a nasal cannula delivery device.

The use of a nasal cannula delivery device may serve useful medical purposes. A nasal cannula delivery device may deliver a medically prescribed, controlled mixture of gases directly to the nostrils of the user. For example, some individuals, because of health issues, require additional oxygenation using an oxygen nasal cannula delivery device.

A nasal cannula delivery device typically comprises tubing that is at least partially supported in place by being placed along the back of the user's ear or ears. The tubing is then run from the ears out to the front of the user's face to the nose, where the controlled gas mixture may be delivered to the user's nostrils for inhalation. The wearing of the nasal cannula delivery device in this fashion allows the user to minimize the level of interference that the wear and use of the device causes with daily activities.

However, many users who wear nasal cannula delivery devices in this fashion have experienced sores and/or skin ulcers (mainly to the tissues around their ears) directly resulting from prolonged skin-to-cannula contact. These injuries can make wearing the nasal cannula delivery device very uncomfortable and/or painful, and can invite infection. Injuries from the use of a nasal cannula delivery device can, in some cases, become very serious. Further, these injuries can cause increased medical and legal costs for the parties involved in the use, manufacture, sale, and provision of nasal cannula delivery devices.

Accordingly, the present disclosure includes an apparatus for holding the nasal cannula delivery device off of the areas around the ears that are most typically irritated or injured through the use of a nasal cannula delivery device. According to one embodiment, the apparatus may be an adjustable piece of headgear that provides cushioning that holds the cannula tubing up and off of the tissues around the ears of the user, while still enabling the ears to provide the necessary support to the cannula tubing so as to enable the normal or selective positioning of the nasal cannula delivery device.

According to various embodiments, the apparatus includes a flexible material that is capable of conforming to the user's head. By way of example and not by limitation, the material may be cloth (e.g., flannel, cotton, polyester, or other appropriate cloth), plastic, rubber, or some other flexible material appropriate for wear over or around the outside of the ear. Further, according to various embodiments, the material is appropriate for the attachment (or alternatively, shaping) of various fastener features.

According to various embodiments, the apparatus includes at least one strap. The at least one strap may include a hold piece that attached to the strap in at least one location. The at least one strap is configured and shaped such that it may be worn behind or around at least a portion of the ear. The at least one strap is further configured such that the tubing from a nasal cannula delivery system may be held in place between the hold piece and the strap in order to protect the ear wearing the strap from direct contact with the tubing.

Fasteners of varying size, shape, purpose, and type may be attached to or shaped into various locations on the apparatus. As used in this application, “fastener” means two or more complimentary features that are meant to be used together such that the at least two complimentary features may be optionally joined together (“fastened”) or optionally un-joined from each other (“unfastened”). Complimentary features may individually include components added to or added onto the apparatus, or complimentary features may individually include a modification of the apparatus material (e.g., cutting the material to create a button-hole feature). By way of example and not by way of limitation, a single fastener may include a piece of hook tape placed at one location on the apparatus with a piece of loop tape placed at another location on the apparatus, or a single fastener may include one or more buttons sewn onto one location on the apparatus and one or more corresponding button-holes cut into the body of the apparatus in another location. There is no limit (other than physical space) on the number of fasteners that the apparatus may comprise or on the number of features that any given fastener may comprise. It is further contemplated that a single headgear apparatus (and/or a piece thereof) may exhibit any number of different types of fasteners.

According to various embodiments, fasteners may be used to fasten together a point on a hold piece to a point on a strap. Further, according to various embodiments, fasteners may be used to fasten together multiple straps.

According to some embodiments, the apparatus is configured to hold in place and support tubing behind or around at least a portion of both ears of a user through the use of at least two separate straps. These straps may then be adjustably fastened together to create a single headgear apparatus. The straps may be configured in such a way that fastening them together causes the apparatus to take an appropriate shape and size to cradle the user's head as shaped areas of the two straps are worn around each of the user's ears. This cradling may give the apparatus additional support and stability and aid the apparatus to remain in place. The shape and size of the apparatus may, under various embodiments, be adjustable by adjusting the fastener between the straps of the apparatus.

Alternative embodiments are contemplated wherein the apparatus includes only a single strap. In such embodiments, it may only be desirable to lift the cannula tubing off of a single ear of the user. This may be because the user is wearing a cannula device that only rests upon one ear during the course of normal use. In this case, a single strap may be worn; it may be held in place simply through the weight of the cannula device pressing the device down upon the ear.

As used herein, the designators “hold piece” and “strap” are used for convenience in communication only and are not used by way of limitation. These designations are not intended to limit the pieces they label to any particular shape, size, or material. It is contemplated that these pieces may be any shape or size (even relative to each other) that otherwise fits within the boundaries of this disclosure.

The embodiments of the disclosure are best understood by reference to the drawings. It will be readily understood that the components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure, as claimed, but is merely representative of possible embodiments of the disclosure. In other instances, well-known structures, materials, or operations are not shown or described in detail to avoid obscuring aspects of this disclosure. In addition, the steps of a method do not necessarily need to be executed in any specific order, or even sequentially, nor need the steps be executed only once, unless specifically stated.

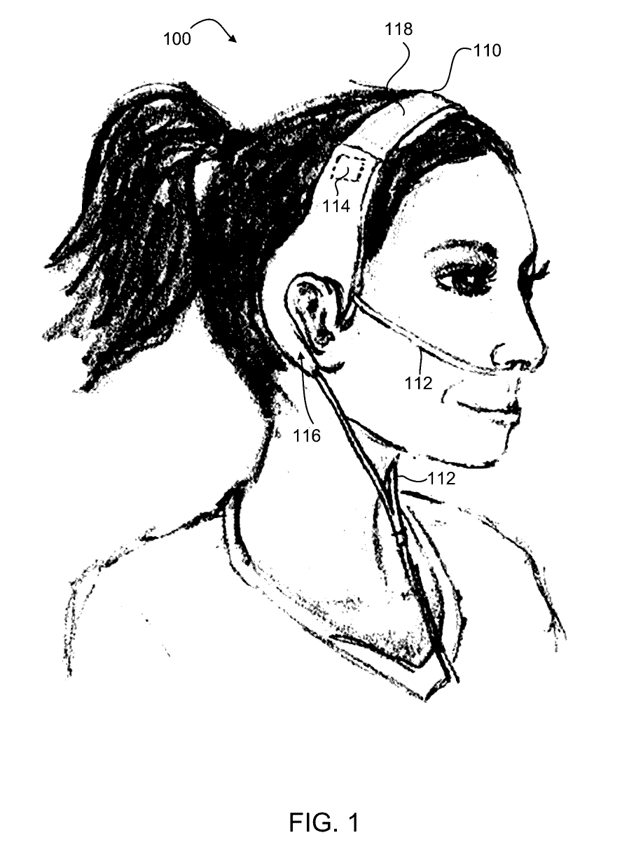

FIG. 1 is a perspective view of a user 100 wearing a headgear apparatus 110 according to one embodiment. The headgear apparatus 110 is designed to help alleviate pain and sores caused by wearing the oxygen delivery device, such as the illustrated oxygen cannula 112. The headgear apparatus 110, according to certain embodiments comprises soft, lightweight, washable flannel.

The headgear apparatus 110 includes a first fastener (not shown) that in some embodiments may be located internally to the headgear in location 118. In some embodiments, the first fastener may include a length of hook tape and a length of loop tape. The first fastener is position-adjustable so that the apparatus can be personalized to the user's desired fit. Each end 116 (one shown) of the headgear apparatus 110 is shaped and configured to comfortably fit around the user's ear. The cannula 112 is placed inside the apparatus 110 in the normal over-ears position, and a second fastener (not shown), which in one embodiment comprises a piece of hook tape and a piece of loop tape and which are located in area 114, helps hold the cannula 112 in place within the headgear apparatus 110. The tension provided by the cannula 112 helps hold the cannula 112 off the ears and keeps the headgear apparatus 110 on the head.

In use, the headgear apparatus 110 lifts the oxygen cannula 112 off of the user's ears so that the cannula 112 is not directly touching the tissues surrounding the ears while maintaining the intended purpose of the cannula 112. This helps prevent pain and pressure sores in the tissues surrounding the ears. The headgear apparatus 110 may also absorb perspiration from the skin.

The headgear apparatus 110 may comprise materials that, when worn, are minimally noticeable by the user 100. By simply putting on a hat or scarf, for example, the headgear apparatus 110 may be less conspicuous when worn in public.

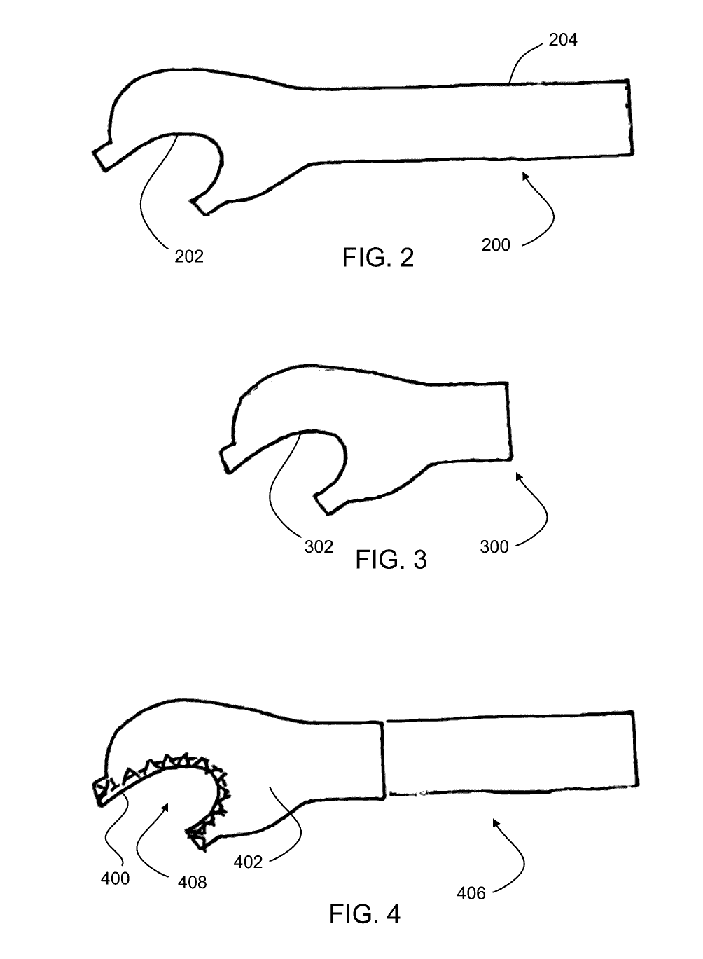

FIG. 2 illustrates a strap 200 according to one embodiment. The strap 200 includes a shaped end 202 that allows for the placement of the strap 200 around at least a portion of the user's ear. In certain embodiments, the shaped end 202 includes a hold fastener (not shown in FIG. 2) that, when disengaged, allows for placement or removal of an ear-worn device such as a cannula and, when engaged, holds the ear-worn device in place. The hold fastener may connect, for example, a fold in the material to form a pocket at the shaped end 202 or a hold piece (e.g., as shown in FIGS. 3-8). The strap also includes an elongated end 204 that, in certain embodiments, includes a first portion of an adjustable strap fastener (not shown in FIG. 2).

FIG. 3 illustrates a hold piece 300 according to one embodiment. The hold piece 300 includes a shaped end 302 that allows for the placement of the hold piece 300 around at least a portion of the user's ear.

The shapes of the strap 200 shown in FIG. 2 and the hold piece 300 shown in FIG. 3 have been illustrated by way of example and not by way of limitation. Persons skilled in the art will recognize that only two of many appropriate, usable shapes have been shown in relation to these pieces, and that both the strap 200 and the hold piece 300 may be shaped in any manner that allows for placement around at least a portion of the user's ear. Persons skilled in the art will also recognize that, while FIGS. 2 and 3 show pieces with substantially similar shapes, this similarity in shape is not necessary in order to create a functional headgear apparatus as disclosed herein. It is contemplated that a strap and a hold piece may be shaped substantially differently relative to each other.

FIG. 4 illustrates a strap 406 according to one embodiment. The strap 406 includes a hold piece 402 attached thereto via attachment 400. As shown, the attachment 400 is performed in a manner such that the shaped ends of the hold piece 402 and strap 406 substantially align, effectively creating one aligned shaped end 408 for the strap 406. This allows for the placement of the strap 406 over the ear of the user in a manner similar to that of the manner of placement over the ear that is possible of a single hold piece 402 or a single strap 406. Further, according to one embodiment, the attachment of the hold piece 402 to the strap 406 takes place along the aligned shaped end 408 of the strap 406. This allows the body of the hold piece 402 and the body of the strap 406 to be optionally separated from each other in order to place nasal cannula tubing along the inside seam of the attachment 400 and in between the hold piece 402 and the strap 406.

FIG. 4 (and other figures herein) shows that the attachment 400 along the shaped ends of the hold piece 402 and the strap 406 may be performed by, for example, sewing. Persons skilled in the art will recognize that the attachment of the hold piece 402 to the strap 406 may also comprise a gluing, a melting together of the respective pieces, or any other method appropriate for attaching together multiple pieces of flexible material along substantially aligned shaped ends. Persons skilled in the art will also recognize that the attachment together of multiple pieces of flexible material may occur before or while the pieces are being shaped.

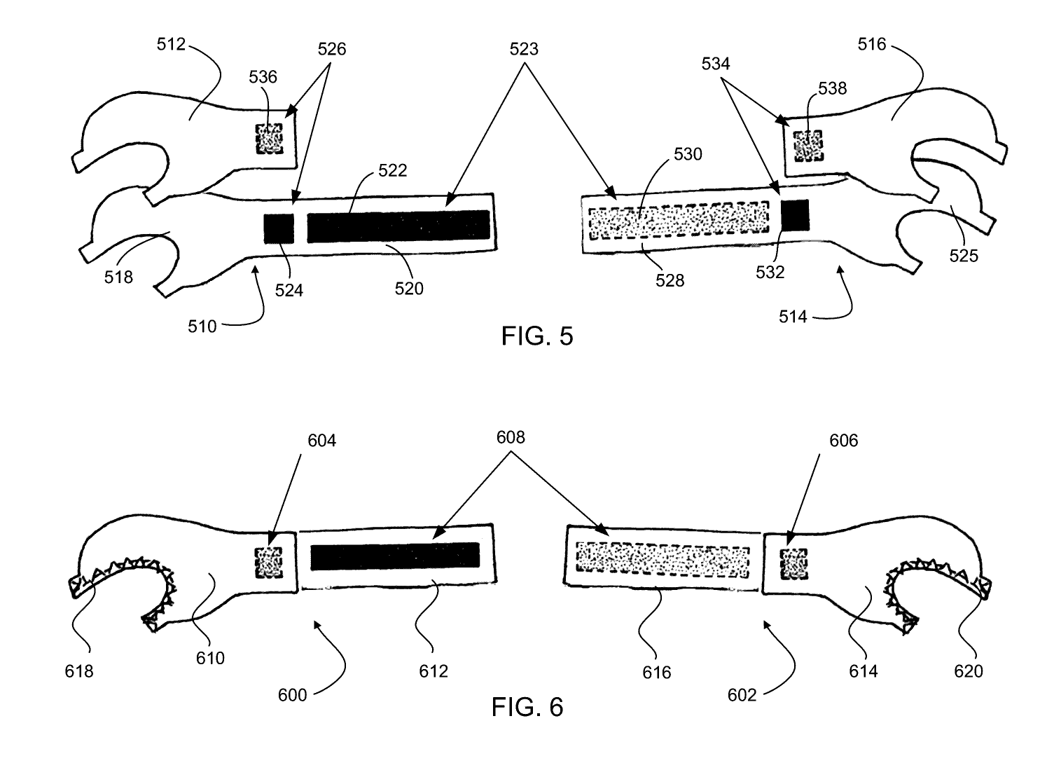

FIG. 5 illustrates a top view of a first strap 510, a first hold piece 512, a second strap 514, and a second hold piece 516, which each comprise a flexible material, according to one embodiment. The first strap 510 includes a shaped end 518 and an elongated end 520. A first portion 522 of an adjustable strap fastener 523 is attached along a top side of the elongated end 520 of the first strap 510. A first portion 524 of a first hold fastener 526 is attached between the shaped end 518 and the elongated end 520 on the top side of the first strap 510.

The second strap 514 includes a shaped end 525 and an elongated end 528. Although not normally visible from the top view, for illustrative purposes a second portion 530 of the adjustable strap fastener 523 is shown attached along a bottom side of the elongated end 528, and a first portion 532 of a second holder fastener 534 is shown attached between the shaped end 525 and the elongated end 528 on the bottom side of the second strap 514.

Also for illustrative purposes, a second portion 536 of the first hold fastener 526 is shown attached to a bottom side of the first hold piece 512, and a second portion 538 of the second holder fastener 534 is shown attached to a bottom side of the second hold piece 516.

FIG. 5 illustrates relative placements of the components of the hold fasteners 526, 534 and the adjustable strap fastener 523 of a functional apparatus according to one embodiment. FIG. 5 shows hold pieces 512, 516 as not yet attached to their respective straps 510, 514. Persons skilled in the art will recognize that the attachment of any hold fastener and/or adjustable strap fastener feature to any part of the apparatus may occur before, after, or during the attachment together of any hold piece and strap pair and/or before, during, or after the shaping of the various parts of the apparatus.

FIG. 6 illustrates two completed straps 600 and 602, including the respective placement of hold fasteners 604 and 606, and components of an adjustable strap fastener 608, according to one embodiment. Regarding the strap 600, the body of the hold piece 610 may optionally be lifted away from the body of the strap 600 after unfastening the hold fastener 604. Similarly, the body of hold piece 614 may optionally be lifted away from the body of the strap 602 after unfastening the hold fastener 606.

Alternatively, the body of the hold piece 610 and the body of the strap 600 can optionally be joined together by fastening the hold fastener 604. Similarly, the body of the hold piece 614 and the body of the strap 602 can optionally be joined together by fastening the hold fastener 606. With their respective hold fasteners 604 and 606 fastened, the straps 600 and 602 form respective pockets that may each hold cannula tubing in place along the seams of their respective attachments 618 and 620 (e.g., shown as stitching) between their respective hold pieces 610 and 614 and straps 600 and 602.

According to some embodiments, the components of the adjustable strap fastener 608 are located on elongated portions 612, 616 of the straps. This placement may be chosen such that the adjustable strap fastener 608 does not interfere or overlap with other fasteners. The placement of the adjustable strap fastener 608 may be done in such a way that fastening the straps 600, 602 together is positionally adjustable, allowing for variation in the relative orientation between the two straps 600, 602 and/or the overall length of the apparatus. In the embodiment shown in FIG. 6, the ability to positionally adjust the fastening between the two straps 600, 602 allows the user to size the apparatus to a desirable position, such as a position appropriate to snugly fit the apparatus against the user's head when worn on the ears.

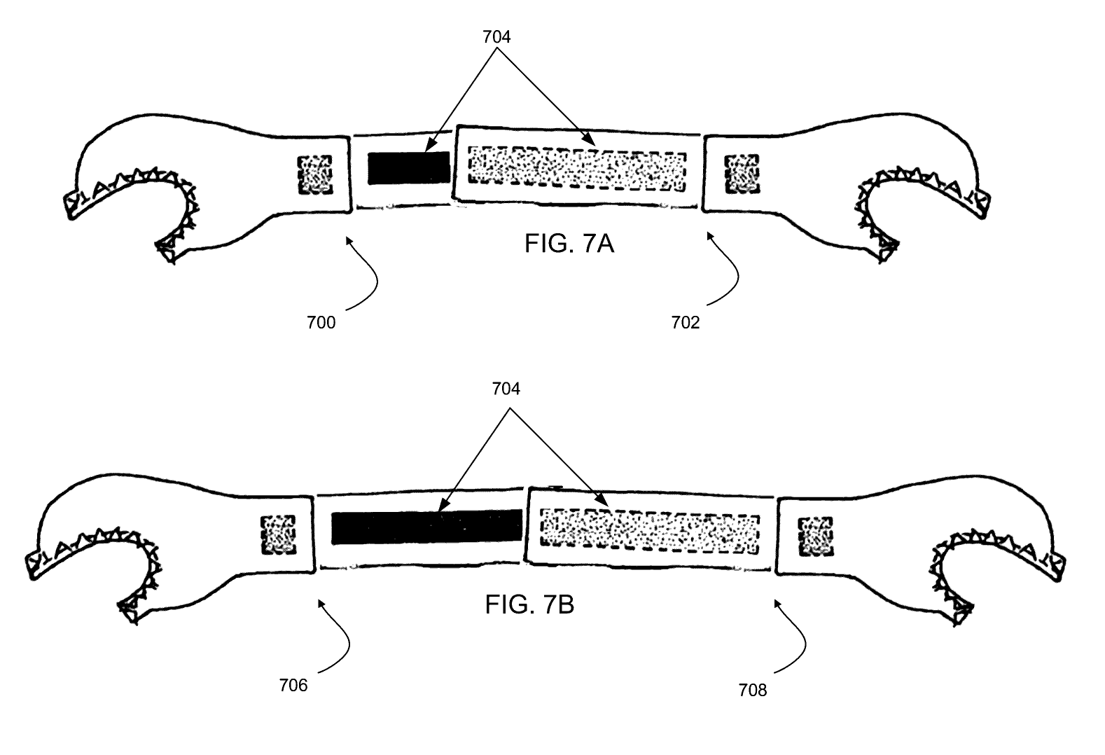

For example, FIG. 7A illustrates the manner in which straps 700 and 702 may be fastened in a positionally adjustable manner according to one embodiment. An adjustable strap fastener 704, according to one embodiment, is comprised of pieces of hook and loop tape attached to the straps 700 and 702, which pieces of hook tape and loop tape a user has pressed together at a certain location in order to create a fully connected headgear apparatus of a first desired length. The straps 700 and 702 may be fastened via the adjustable strap fastener 704 in a linear manner, or they may be allowed to be fastened via the adjustable strap fastener 704 in any other orientation relative to each other. For example, fastener 704 in FIG. 7A has been fastened such that the straps 700 and 702 are not precisely linear with each other.

FIG. 7B illustrates a manner in which the straps 706 and 708 may be alternatively connected in a positionally adjustable manner by adjusting the embodiment of FIG. 7A. In FIG. 7B, the user (or a different user) has fastened the fastener 704 in a different position than the position of the fastener 704 shown in FIG. 7A in order to create a fully connected headgear apparatus of a longer length.

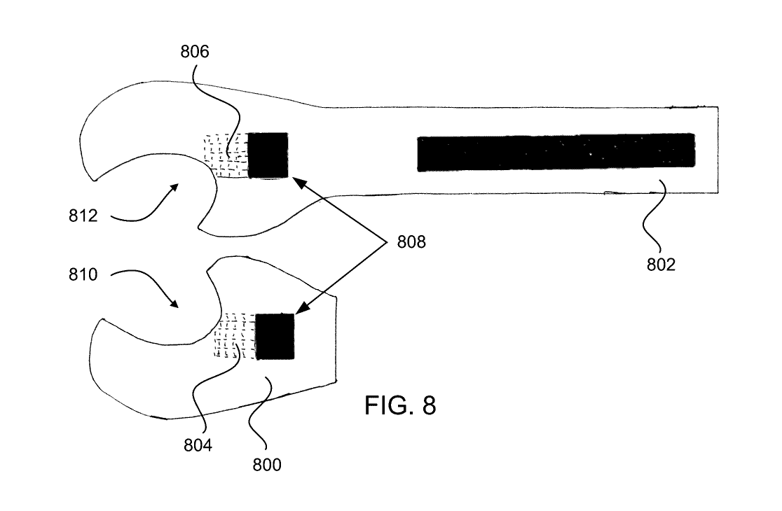

FIG. 8 illustrates another embodiment of both a hold piece 800 and a strap 802. For illustrative purposes, FIG. 8 shows a top side of the strap 802 and a bottom side of the hold piece 800. In this embodiment, pieces of non-slip material 804 and 806 have been added to the hold piece 800 and the strap 802, such that when the hold piece 800 and the strap 802 are attached along their respected shaped ends 810 and 812, the tubing inserted into the strap is held along the connected seam created by the attachment of shaped ends 810 and 812 by fastener 808, and is substantially prevented from sliding within the confines of the strap due to the friction of the tube's contact with the non-slip material pieces 804 and 806.

Reference throughout this specification to “one embodiment” or “an embodiment” means that a particular feature, structure, or characteristic described in connection with the embodiment is included in at least one possible implementation. Thus, the phrases “in various embodiments” and “in some embodiments” appearing throughout this specification are not necessarily all referring to the same embodiment. However, all possible combinations and permutations of each of the described embodiments are contemplated as possible embodiments.

The above description provides numerous specific details for a thorough understanding of the embodiments described herein. However, those of skill in the art will recognize that one or more of the specific details may be omitted, modified, and/or replaced by a similar process, system, or component. In many instances the order of steps and/or actions of the methods of use described herein may be interchanged with one another.

This disclosure has been made with reference to various embodiments, including the best mode. However, those skilled in the art will recognize that changes and modifications may be made to the exemplary embodiments without departing from the scope of the present disclosure. While the principles of this disclosure have been shown in various embodiments, many modifications of structure, arrangements, proportions, elements, materials, and components may be adapted for a specific environment and/or operating requirements without departing from the principles and scope of this disclosure. These and other changes or modifications are intended to be included within the scope of the present disclosure.

It will be understood by those having skill in the art that many changes may be made to the details of the above-described embodiments without departing from the underlying principles of the invention. The scope of the present invention should, therefore, be determined only by the following claims.

1. An apparatus for separating an ear-worn device from skin of a user, comprising:
a first strap comprising flexible material, the first strap including a first shaped end configured for placement around a portion of a first ear of the user, and a first elongated end comprising a first portion of an adjustable strap fastener; and
a second strap comprising flexible material, the second strap including a second shaped end configured for placement around a portion of a second ear of the user, and a second elongated end comprising a second portion of an adjustable strap fastener;
wherein at least one of the first shaped end and the second shaped end comprises a hold fastener that, when disengaged, allows for placement or removal of the ear-worn device and, when engaged, holds the ear-worn device in place; and
wherein the first portion of the adjustable strap fastener and the second portion of the adjustable strap fastener are configured to engage and disengage each other to selectively adjust the size of the apparatus to fit a head of the user.

2. The apparatus of claim 1, further comprising a first hold piece attached to an edge of the first shaped end of the first strap, the first hold piece configured for placement around the portion of the first ear of the user, wherein a first portion of the hold fastener is affixed to the first strap and a second portion of the hold fastener is affixed to the first hold piece to allow the body of the first strap and the body of the hold piece to be selectively connected by the hold fastener for the placement or removal of the ear-worn device.

3. The apparatus of claim 2, further comprising a second hold piece attached to an edge of the second shaped end of the second strap, the second hold piece configured for placement around the portion of the second ear of the user.

4. The apparatus of claim 2, wherein the first strap, the second strap, and the first hold piece comprise flannel.

5. The apparatus of claim 2, wherein the first strap, the second strap, and the first hold piece comprise plastic.

6. The apparatus of claim 2, wherein the straps are comprised of cotton and the hold pieces are comprised of rubber.

7. The apparatus of claim 2, wherein the first strap comprises a first piece of non-slip material and the first hold piece comprises a second piece of non-slip material, wherein the first piece of non-slip material is aligned with the second piece of non-slip material when the body of the first strap and the body of the hold piece are connected by the hold fastener to reduce movement of the ear-worn device when held by the apparatus.

8. The apparatus of claim 1, further comprising one or more pieces of non-slip material attached to at least one of the first strap and the second strap such that the non-slip material contacts the ear-worn device when the ear-worn device is held by the apparatus with the hold fastener engaged.

9. The apparatus of claim 1, wherein the hold fastener comprises hook tape and loop tape.

10. The apparatus of claim 1, wherein the hold fastener comprises a button and a button-hole.

11. The apparatus of claim 1, wherein the hold fastener comprises a snap connector nub and a snap connector receiver.

12. The apparatus of claim 1, wherein the adjustable strap fastener is comprised of hook tape and loop tape.

13. The apparatus of claim 1, wherein the adjustable strap fastener comprises a snap fastener nub and a snap fastener nub receiver.

14. The apparatus of claim 1, wherein when the first portion of the adjustable strap fastener and the second portion of the adjustable strap fastener are configured to selectively adjust the size of the apparatus to fit a head of the user and the apparatus is configured to fit snugly against the user's head such that the first shaped end fits around the first ear and the second shaped end fits around the second ear.

15. A method of creating an apparatus for separating an ear-worn device from the skin of a user, comprising:
attaching one of each of at least two straps of a flexible material to one of each of at least two hold pieces of flexible material, wherein each strap and each hold piece are shaped in at least one location to allow for placement around at least a portion of the user's ear, and wherein the attachment is performed such that the shaped ends of each attachment of hold piece and strap substantially align;
locating fasteners on each of the at least two straps such that a body of a hold piece may be optionally joined together with a body of its attached strap or optionally un-joined from and lifted away from the body of its attached strap, wherein the fastener is located such that there is sufficient room to facilitate the ear-worn device in between the location of the attachment and the location of the fastener when fastened;
locating at least one complementary feature of at least one fastener on each of the at least two straps such that the at least two straps may be optionally fastened together.

16. The method of claim 15, additionally comprising locating one or more pieces of non-slip material on one or more straps such that the pieces of non-slip material contact the ear-worn device when the ear-worn device is placed inside a strap with a subsequently fastened hold fastener.

17. The method of claim 15, where at least one attachment of a strap to a hold piece is performed by sewing.

18. The method of claim 15, where at least one attachment of a strap to a hold piece is performed by gluing.

19. An apparatus for separating an ear-worn device from the skin of a user, comprising:
a strap of a flexible material shaped in one location to allow for placement of the strap around at least a portion of the user's ear;
a hold piece of flexible material shaped in one location to allow for placement of the hold piece around at least a portion of the user's ear;
wherein the hold piece is attached together with the strap, the attachment occurring along or near to the shaped ends of the hold piece and the strap such that the respective shaped ends of the hold piece and the strap substantially align;
wherein the strap is fitted with a hold fastener that, when disengaged, allows the placement of the ear-worn device between the strap and hold piece and, when engaged, holds the ear-worn device in place between the strap and the hold piece.

20. The apparatus of claim 19, wherein at least one of the strap and the hold piece comprises one or more flexible material selected from a group comprising flannel, cotton, polyester, and rubber.
